(19) United States
(12) Patent Application Publication
(10) Pub. No.: US 2009/0312293 A1
(43) Pub. Date: Dec. 17, 2009

(54) PROCESS FOR THE ISOLATION OF PHARMACOLOGICALLY ACTIVE PRINCIPLES OF VEGETABLE AND ANIMAL ORIGIN

(75) Inventors: Giancarlo Mazzola, Medelia (CH); Anna Anzani, Milano (IT)

Correspondence Address: YOUNG & THOMPSON, 209 Madison Street, Suite 500, Alexandria, VA, 22314, US

(73) Assignee: EVULTIS S.A., Lugano (CH)

(21) Appl. No.: 12/159,212

(22) PCT Filed: Dec. 19, 2006
(86) PCT No.: PCT/EP2006/012244

§ 371 Date: Feb. 10, 2009

(30) Foreign Application Priority Data
Dec. 29, 2005 (IT) MI2005A002516

(51) Int. Cl. A61K 31/566; A61K 31/565; C07J 1/00

(52) U.S. Cl. 514/170; 549/403; 552/625

(57) ABSTRACT

A process for the recovery and purification of natural hydrophilic water-soluble products in conjugated form from vegetable aqueous extracts or physiological fluids, by adsorption of the extracts or fluids on a lipophilic resin, followed by desorption and recovery of the eluate, which process is characterized in that the resin is a porous styrene-divinyl benzene polymer brominated at the styrene and/or divinylbenzene portion, with 600 m²/g area, 1.3 ml/g volume (dry weight), about 200 Angstrom pore size.

The invention relates to a process for the isolation and purification of pharmacologically interesting natural compounds from animal or vegetable sources.

More particularly, the invention relates to the extraction of hydrophilic and water-soluble compounds present in said sources in a form conjugated to inorganic acids, e.g. the form of sulfates, or in glycosylated form.

The process comprises the adsorption of the sources containing said compounds on a highly lipophilic resin, followed by desorption and recovery of the eluate.

Preferred examples of compounds obtainable by process of the invention comprise phytoestrogens (isoflavones), estrogens completely conjugated to steroidal, polyphenolic structures of animal or vegetable origin having various therapeutical or preventive pharmacological activities, such as antioxidant and antitumor activity.

The compounds obtainable by the process of the invention in high purity and reproducibility are useful for the preparation of medicaments or food supplements, for the treatment of both primary pathologies such as uterus and prostate tumors, and acute or chronic inflammations, and pathologies deriving from paraphysiological conditions such as premenstrual syndrome and tension, osteoporosis and atherosclerosis related with aging and/or hormonal alterations.

### TECHNOLOGICAL BACKGROUND

A number of food supplements based on phytotherapic extracts exist, whose composition and reproducibility markedly change, depending on the extraction techniques used. Those changes mainly concern the presence of products different than those desired. As a consequence, said extracts cannot be used as medicaments, as they do not fulfil the mandatory requirements of uniform composition and absence of other components.

WO 93/23069, WO 99/43335 and EP 1174144 disclose soy or clover extracts containing mixtures of genistein, daidzein, formononetin and biochanin A.

EP 1174144 claims a process for the extraction of isoflavone aglycones, in particular from Trifolium pratensis, by maceration of the dried, finely powdered vegetable material in water, followed by extraction by addition of a water-miscible solvent (usually ethanol); separation of the vegetable residue; treatment of the aqueous solution with an aliphatic hydrocarbon to remove waxes and fats; separation of the phases and removal of the hydrocarbon one; distillation of the water-miscible organic solvent in vacuo, to give a solid (water-insoluble isoflavone aglycones) which is filtered and dried. Aglycones content assayed by HPLC ranges from 19.5% to 38% w/w, while yields range from 0.6% to 2.2% based on the starting material.

WO 93/23069 discloses compositions enriched in isoflavone phytoestrogens obtained by extracting the dry vegetable material with a water: water-soluble organic solvent (e.g. ethanol) mixture, separating the water-organic extract, distilling off the organic solvent and concentrating the aqueous phase.

WO 99/43335 discloses the preparation of isoflavones-containing clover extracts characterized by the presence of “aromatic chromophores” (genistein, daidzein, formononetin and biochanin-A). The extraction procedure is substantially the same as described in WO 93/23069, although containing a further purification/isolation step of the isoflavones by HPLC.

In case of animal physiologic fluids such as urine from pregnant animals, processes are described for the preparation of conjugated estrogens mixtures by use of different semipolar, but markedly hydrophilic, non-ionic adsorbent resins, such as Amberlites®, Diaion Sepabeads® or HPD-500®. For example, U.S. Pat. No. 5,723,454 discloses a method for the extraction of conjugated estrogens from pregnant mare urine in which urine previously clarified by filtration through a sand bed, by centrifugation or by ultrafiltration, are exposed (by contact in suspension or by percolation on a column) to Amberlite® non-ionic resins (cross-linked polyacrylic esters, e.g. XAD-7 by Rohm & Haas), with medium polarity (dipolar moment ranging from 1.5 to 2.0 Debye) and specific areas from 400 to 500 m²/g. The resin is subjected to repeated washings with alkali water, then the estrogen mixture is recovered by elution with an alkali water: miscible organic solvents mixture and obtained in the solid form by concentration in vacuo and drying.

US 2005/0014738, US 2003/0105344 and US 2004/0072812 disclose the same adsorption/desorption process of conjugated estrogens, again using medium-polarity, mainly hydrophilic resins such as Dowex® XAD2, Dowex®, Optipore. US 2002/0156303 describes the use of Diaion® HP-20 and Sepabeads® SP-700 (Mitsubishi) resins, also having medium polarity. CN 1308038 makes use of the semi-polar resin HPD-500 manufactured by Hebej Cangzhou Chemical Factory, having similar characteristics. The contact and extraction steps of the absorbed material are also similar.

All of said methods are based on the use of non-ionic, medium-polarity, mainly hydrophilic resins, which have poor adsorption selectivity, thereby yielding mixtures of conjugated and unconjugated estrogens, as well as noticeable amounts of cresol derivatives. This involves a series of purifications steps following the adsorption and sometimes even the elimination of the worked batch due to the unacceptable content in unconjugated estrogens or other polyphenolic impurities.

It is therefore evident the need for reproducible processes which overcome the above mentioned problems, making the composition of both animal and vegetable origin conjugates constant and free from unconjugated products or other impurities.

### DISCLOSURE OF THE INVENTION

It has now surprisingly been found that the use of a highly porous styrene-divinyl benzene polymer, with an area 600 m²/g, volume 1.3 ml/g (dry weight), size of pores about 200 Angstrom, characterized by the bromination of one of the two polymeric components, provides the purification of the compounds mentioned above in high yields and the use thereof in the pharmaceutical field.

The process of the invention can be applied both to “primary extracts”, defined as crude starting liquid or solid extracts, obtained from vegetable or animal sources with known methods, and to physiological fluids.

Examples of products of vegetable origin include isoflavones or lignans, such as genistein, daidzein, formononetin, biochanin A, coumestrol, ferulic and isoferulic acids, present in various vegetable sources in the monosaccharide-conjugated, water-soluble form.

Examples of products extractable from physiological fluids include steroidal estrogens such as estrone and estradiol present in pregnant female urine, in which they are conjugated with inorganic acids. In particular, pregnant mare urine provides a mixture of conjugated estrogens salts comprising estrone, equilin, Δ^{8,9}-dehydroestrone, 17α-estradiol; 17α-dihydroequilin, 17β-dihydroequilin, 17β-estradiol, equilenin, 17α-dihydroequilenin; 17β-dihydroequilenin and optionally also one or more conjugated salts from the group of 17β-Δ^{8,9}-dehydroestradiol; 17 α-Δ^{8,9}-dehydroestradiol; 6-OH 17α-dihydroequilenin; 6-OH equilenin; 6-OH 17β-dihydroequilenin and/or other sulphated steroidal metabolites. Salts are preferably sodium salts, while conjugated are mainly sulfates.

Resin bromination involves an increase in the particles specific weight (which allows, inter alia, to work in column through both direct percolation and expanded bed) and induces less polymer hydration, hence lower polar charge and much higher lipophilia than other non-brominated styrene-divinylbenzene polymers used to date. Brominated resins having such characteristics are manufactured by Mitsubishi Chem. Co, for example: Diaion SP 207®, Diaion SP 205®, Diaion SP 206®.

The results obtained according to the invention are unexpected in the light of the markedly hydrophilic and reduced lipophilia characteristics of the products to purify. The behaviour of these products with respect to the adsorption on mainly lipophilic resins, such as the above resins, which are more specific for lipophilic molecules, is therefore surprising and unexpected.

The process of the invention comprises the following steps:

adsorption, in bulk or on a column, of said primary extracts or fluids, optionally clarified by pre-filtration, centrifugation or ultrafiltration, by direct percolation or expanded bed (i.e. from the bottom of the column) on said brominated styrene-divinyl benzene polymers, with consequent adsorption of the active principles or mixtures thereof and any impurities;

in case of work up in bulk, separation and squeezing (by in vacuo filtration or centrifugation) of the adsorption solid phase from the liquid, which is discarded;

selective desorption of the adsorbed products, active principles and impurities in column, by means of gradients (either concave or convex) of water:water-miscible solvents and optionally of pH;

recovery of the eluates and drying in vacuo;

optional further purification and/or crystallization steps of the solid residues.

The contact between resin and fluid or primary extract can be carried out either in bulk, keeping the heterogeneous mass under slow stirring (to avoid pulverization of the resin) or in chromatographic column, by percolation or expanded bed techniques.

The selection of the process depends on both the type and amount of worked product. In case of in bulk technique, the preferred stirrer is that a blade stirrer. The resin will be used in amounts depending on the nature of the fluid or primary extract to process and the content in active principles, as assayed by conventional analyses (HPLC, GC) but, as a rule, production performance being the same, this amount is approximately 60-75% of the above semi-polar resins. In case of work up in bulk, three- to four-fold amounts than those for column are used. The solution to adsorb can be clarified with known techniques, for example by filtration on a sand bed or centrifugation in suitable apparatus for the recovery of the fluid by ultrafiltration. In case of work up in bulk, the adsorbed resin is recovered, squeezed from the excess fluid by filtration in vacuo and placed in a column fitted with porous septum and cooling jacket. The adsorbate is eluted with a water: miscible solvents mixture (e.g. 70-30 v/v water-ethanol), and adjusted with sodium hydroxide to alkali pH from 11 to 13, preferably from 11 to 12.5. The optionally concentrated crude eluate is then purified by chromatography on the same brominated resin as used for the primary extraction, or on other brominated resins, following the procedure described for the chromatographic direct work up. The process in bulk is particularly advantageous for the preparation of vegetable origin derivatives.

In case of work up with chromatographic techniques, which is preferred for fluids from animal sources, the optionally concentrated primary solution is passed on a resin bed, by either percolation or expanded bed. The latter technique is favoured by the high specific weight of the particles of the brominated resins and it advantageously exposes the solution to a larger adsorbing surface, as the slight overpressure separates the porous microbeads from each other, which conversely form a more compact bed when using direct percolation. The application of a slight overpressure to the supplied solution increases the adsorption yield and reduces operative times. The resins that can be used in the adsorption process are highly porous brominated styrene-divinyl benzene polymers, such as Diaion SP 207®, Diaion SP 205® and Diaion SP 206® manufactured by Mitsubishi Chemical Co. Diaion SP 207® is preferred. The ratio of resin to solution to treat can range from 1 part by volume of resin per 25 parts of solution to 1 part of resin per 200 parts of solution, depending on the type of SP used and the nature of the active principle(s) to extract. More particularly, in case of Diaion SP 207, said ratio can range from 25 to 150. Elution is monitored with UV detection between 270 and 280 nm. The volume of washing liquids for removing the adsorbed solution still in the void of the column is usually 1.8 to 2-fold the column bed. Washing liquids have composition varying according to the nature and the amount of the impurities and the type of adsorbed product. Temperatures range from 0° C. to 35° C., preferably from 0° C. to 5° C. The preferred washing liquid is water, but small amounts (1-5%) of water-miscible solvents (e.g. acetone or ethanol) can also be present.

The adsorbed product is eluted with water:water-miscible solvents (e.g. ketones, low molecular alcohols, water-soluble ethers or esters) mixtures whose composition can range from 100% to 0.10% water/solvent, depending on the nature of the impurities and the origin of the material. The mixtures are adjusted to pH 11-13, preferably 11-12.5, with sodium hydroxide. Elution is monitored through the UV absorption between 270 and 280 nm. Fractions are analyzed and those containing the desired compounds are combined and concentrated in vacuo.

The following examples illustrate in detail the invention.

### EXAMPLE 1

### Preparation of Conjugated Isoflavones By Direct Extraction of Trifolium Pratensis

500 g of dried, finely ground Trifolium pratensis are treated with 2000 ml of distilled water under stirring at r.t. for 10 hours. The solid is then filtered and the solution is analyzed by HPLC for the content in conjugated and unconjugated isoflavones. The conjugated isoflavones total content is approximately 500 mg, free aglycones being substantially absent. The solution is concentrated in vacuo to 250 ml, further filtered, then percolated onto a column of 1-1.5 cm of diameter packed with 10 g of Sepabeads 207® Mitsubishi, collecting the fractions and monitoring the elution by UV at 270 nm. After completion of the percolation, the column is washed with about twice the void with distilled water, then water containing 5% ethanol (200 ml). The fractions containing the conjugated isoflavones (HPLC analysis) are combined. The solution is filtered, the residue is dried in vacuo (about 420 mg) then taken up with dry acetone to give a solid residue, in about 84% yield on the starting aqueous extract. The residue consists of the glycosides of biochanin-A, formononetin, daidzein and genistein with 98% purity.

### EXAMPLE 2

### Preparation of Isoflavones By Extraction of Trifolium Pratensis Aqueous Extracts

20 g of dry extract obtained according to EP 1174144 (Example 1) with content in free isoflavones of about 20% and in conjugated isoflavones of about 30% (HPLC) are dispersed in 500 ml of water; the suspension is filtered in vacuo. HPLC analysis shows the content in conjugated aglycones is about 28% and in unconjugated aglycones is about 1% on the starting dry weight (unconjugated aglycones are removed, together with other components, due to their insolubility in water). The solution is concentrated to 200 ml by distillation in vacuo and treated with 80 g of adsorbent Sepabeads® SP 207 (Mitsubishi). Stirring is continued for 2 h. The adsorbed resin is then filtered in vacuo, squeezed and placed on a column fitted with porous septum. 1.8 volumes of the column bed are then added with distilled water pre-cooled at 0-5° C. After that, 2.5 times the bed volume are added with a 95/5 water:ethanol mixture, collecting the eluate which is subjected to distillation in vacuo at a temperature not exceeding 40° C. until drying. The solid residue is taken up with acetone, then with acetone/ethyl ether and triturated, solvents are distilled off in vacuo and the solid is recovered. HPLC analysis shows that the product consists of genistein, daidzein, formonetin and biochanin A, and is substantially free from impurities (content in conjugated isoflavones: 90-95%). Yields range from 80 to 85% on the starting extract.

### EXAMPLE 3

### Extraction of Conjugated Estrogens From Pregnant Mare Urine

20 l of pregnant mare urine are filtered first on a sand bed of about 10 cm, then through a 0.2μ membrane. The content in conjugated estrogens is determined by HPLC or GC. pH is adjusted to about 12.5-13.5 by addition of concentrated sodium hydroxide. The whole is kept under mechanical stirring for approx. 1-2 h, under nitrogen. pH is then adjusted to neutrality (pH 7.5-8.5, preferably 8) with a mineral acid, preferably HCl or trifluoroacetic acid. The solution is further filtered in vacuo on sand, then on membrane. The clear filtrate is passed, by either percolation or expanded bed, with slight overpressure so as not to increase the resin bed by more than 3-5% in height in the case of the expanded bed and not to induce packing of the resin when using direct percolation, on a column of diameter from 7.5 and 10 cm packed with 150-180 g of Sepabeads 207® Diaion (Mitsubishi). In the case of the expanded bed, the column bed has to be at least of 30-50 cm. After completion of the elution, the resin is cooled by circulation of liquid cooler at 0°-5° C., then the adsorbate is washed with at least 1.8-2/5 volumes of void with distilled water at 0°-5° C. The bed is then percolated (or expanded) with water at pH 11.5-13.0 by addition of concentrated NaOH, at a temperature of 5°-10° C. (2 and 4 volumes that of the resin). The conjugated estrogens complex is then eluted with a mixture of water: water miscible solvents (acetone, ethanol, THF) in 30:70 minimum ratio, then adjusted to pH 10-13, preferably 12.5-13, by addition of sodium hydroxide. The eluate is recovered, neutralized and dried in vacuo to provide the active ingredient.

1. A process for the recovery and purification of natural hydrophilic water-soluble products in conjugated form from vegetable aqueous extracts or physiological fluids, by adsorption of said extracts or fluids on a lipophilic resin, followed by desorption and recovery of the eluate, which process is characterized in that the resin is a porous styrene-divinyl benzene polymer brominated at the styrene and/or divinylbenzene portion, with 600 m²/g area, 1.3 ml/g volume (dry weight), about 200 Angstrom pore size.

2. A process as claimed in claim 1 wherein natural products comprise isoflavones, antioxidants or conjugated estrogens or mixtures thereof.

3. A process as claimed in claim 2 wherein isoflavones are extracted from soy or Trifolium pratensis.

4. A process as claimed in claim 3 wherein isoflavones comprise genistein, daidzein, formononetin, biochanin A.

5. A process as claimed in claim 1 for the purification and recovery of conjugated estrogens from pregnant mammal fluids.

6. A process as claimed in claim 5 mixture of salts of the physiological fluid is pregnant mare urine.

7. A process as claimed in claim 6 for the preparation of a conjugated estrogens mixture salts mixture comprising estrone, equilin, Δ^{8,9}-dehydroestrone, 17α-estradiol; 17α-dihydroequilin, 17β-dihydroequilin, 17β-estradiol, equilenin, 17α-dihydroequilenin; 17β-dihydroequilenin.

8. A process as claimed in claim 7 for the preparation of a mixture further containing one or more conjugated salts from the group of 17β-Δ^{8,9}-dehydroestradiol; 17α-Δ^{8,9}-dehydroestradiol; 6-OH 17α-dihydroequilenin; 6-OH equilenin; 6-OH 17β-dihydroequilenin and/or other sulfated steroidal metabolites.

9. A process as claimed in claim 6 wherein the conjugates are sulfates and the salts are sodium salts.

10. A process as claimed in claim 1 wherein the resins are selected from those commercially available under the commercial names Diaion SP 207®, Diaion SP 205® and Diaion SP 206®.

11. A process as claimed in claim 2 for the purification and recovery of conjugated estrogens from pregnant mammal fluids.

12. A process as claimed in claim 11 mixture of salts of the physiological fluid is pregnant mare urine.

13. A process as claimed in claim 12 for the preparation of a conjugated estrogens mixture salts mixture comprising estrone, equilin, Δ^{8,9}-dehydroestrone, 17α-estradiol; 17α-dihydroequilin, 17β-dihydroequilin, 17β-estradiol, equilenin, 17α-dihydroequilenin; 17β-dihydroequilenin.

14. A process as claimed in claim 13 for the preparation of a mixture further containing one or more conjugated salts from the group of 17β- Δ^{8,9}-dehydroestradiol; 17α-Δ^{8,9}-dehydroestradiol; 6-OH 17α-dihydroequilenin; 6-OH equilenin; 6-OH 17β-dihydroequilenin and/or other sulfated steroidal metabolites.

15. A process as claimed in claim 7 wherein the conjugates are sulfates and the salts are sodium salts.
